SEQUENCE LISTING (10 sequences)

SEQ ID NO: 1 - protein, 39 aa, synthetic construct
  SITE 2: note = X at position 2 is Aib
  SITE 13: note = X at position 13 is Aib
  MOD_RES 1: note = N-terminus is protected by Boc (t-butyloxycarbonyl)
  MOD_RES 1: note = The sidechain of tyrosine is protected by t-butyl
  MOD_RES 3: note = The sidechain of glutamic acid is protected by t-butyl
  MOD_RES 5: note = The sidechain of threonine is protected by t-butyl
  MOD_RES 7: note = The sidechain of threonine is protected by t-butyl
  MOD_RES 8: note = The sidechain of serine is protected by t-butyl
  MOD_RES 9: note = The sidechain of aspartic acid is protected by t-butyl
  MOD_RES 10: note = The sidechain of tyrosine is protected by t-butyl
  MOD_RES 11: note = The sidechain of serine is protected by t-butyl
  MOD_RES 15: note = The sidechain of aspartic acid is protected by t-butyl
  MOD_RES 16: note = The sidechain of lysine is protected by Boc (t-butyloxycarbonyl)
  MOD_RES 19: note = The sidechain of glutamine is protected by a Trt (Trityl) protecting group
  MOD_RES 24: note = The sidechain of glutamine is protected by a Trt (Trityl) protecting group
  MOD_RES 20: note = The sidechain of lysine is protected by mtt
  MOD_RES 25: note = The sidechain of Tryptophan is protected by Boc (t-butyloxycarbonyl)
  MOD_RES 32: note = The sidechain of serine is protected by t-butyl
  MOD_RES 33: note = The sidechain of serine is protected by t-butyl
  MOD_RES 39: note = The sidechain of serine is protected by t-butyl
  MOD_RES 39: note = C-terminal amino acid is amine is attached to Resin
YXEGTFTSDYSIXLDKIAQKAFVQWLIAGGPSSGAPPPS

SEQ ID NO: 2 - protein, 39 aa, synthetic construct; identical to SEQ ID NO: 1
  MOD_RES 1: note = The sidechain of tyrosine is protected by t-butyl.
  MOD_RES 1: note = N-terminus is protected by Boc (t-butyloxycarbonyl)
  SITE 2: note = X at position 2 is Aib
  MOD_RES 3: note = The sidechain of glutamic acid is protected by t-butyl
  MOD_RES 5: note = The sidechain of threonine is protected by t-butyl
  MOD_RES 7: note = The sidechain of threonine is protected by t-butyl
  MOD_RES 8: note = The sidechain of serine is protected by t-butyl
  MOD_RES 9: note = The sidechain of aspartic acid is protected by t-butyl
  MOD_RES 10: note = The sidechain of tyrosine is protected by t-butyl
  MOD_RES 11: note = The sidechain of serine is protected by t-butyl
  SITE 13: note = X at position 13 is Aib
  MOD_RES 15: note = The sidechain of aspartic acid is protected by t-butyl
  MOD_RES 16: note = The sidechain of lysine is protected by Boc (t-butyloxycarbonyl)
  MOD_RES 19: note = The sidechain of glutamine is protected by a Trt (Trityl) protecting group
  MOD_RES 24: note = The sidechain of glutamine is protected by a Trt (Trityl) protecting group
  MOD_RES 25: note = The sidechain of Tryptophan is protected by Boc (t-butyloxycarbonyl)
  MOD_RES 32: note = The sidechain of serine is protected by t-butyl
  MOD_RES 33: note = The sidechain of serine is protected by t-butyl
  MOD_RES 39: note = The sidechain of serine is protected by t-butyl
  MOD_RES 39: note = C-terminal amine is attached to Resin

SEQ ID NO: 3 - protein, 39 aa, synthetic construct; identical to SEQ ID NO: 1
  MOD_RES 1: note = N-terminus is protected by Boc (t-butyloxycarbonyl)
  MOD_RES 1: note = The sidechain of tyrosine is protected by t-butyl
  SITE 2: note = X at position 2 is Aib
  MOD_RES 3: note = The sidechain of glutamic acid is protected by t-butyl
  MOD_RES 5: note = The sidechain of threonine is protected by t-butyl
  MOD_RES 7: note = The sidechain of threonine is protected by t-butyl
  MOD_RES 8: note = The sidechain of serine is protected by t-butyl
  MOD_RES 9: note = The sidechain of aspartic acid is protected by t-butyl
  MOD_RES 10: note = The sidechain of tyrosine is protected by t-butyl
  MOD_RES 11: note = The sidechain of serine is protected by t-butyl
  SITE 13: note = X at position 13 is Aib
  MOD_RES 15: note = The sidechain of aspartic acid is protected by t-butyl
  MOD_RES 16: note = The sidechain of lysine is protected by Boc (t-butyloxycarbonyl)
  MOD_RES 19: note = The sidechain of glutamine is protected by a Trt (Trityl) protecting group
  MOD_RES 24: note = The sidechain of glutamine is protected by a Trt (Trityl) protecting group
  MOD_RES 25: note = The sidechain of Tryptophan is protected by Boc (t-butyloxycarbonyl)
  MOD_RES 32: note = The sidechain of serine is protected by t-butyl
  MOD_RES 33: note = The sidechain of serine is protected by t-butyl
  MOD_RES 39: note = The sidechain of serine is protected by t-butyl
  MOD_RES 39: note = C-terminal amino acid is amidated as a C-terminal primary amide

SEQ ID NO: 4 - protein, 39 aa, synthetic construct; identical to SEQ ID NO: 1
  MOD_RES 1: note = N-terminus is protected by Boc (t-butyloxycarbonyl)
  MOD_RES 1: note = The sidechain of tyrosine is protected by t-butyl
  SITE 2: note = X at position 2 is Aib
  MOD_RES 3: note = The sidechain of glutamic acid is protected by t-butyl
  MOD_RES 5: note = The sidechain of threonine is protected by t-butyl
  MOD_RES 7: note = The sidechain of threonine is protected by t-butyl
  MOD_RES 8: note = The sidechain of serine is protected by t-butyl
  MOD_RES 9: note = The sidechain of aspartic acid is protected by t-butyl
  MOD_RES 10: note = The sidechain of tyrosine is protected by t-butyl
  MOD_RES 11: note = The sidechain of serine is protected by t-butyl
  SITE 13: note = X at position 13 is Aib
  MOD_RES 15: note = The sidechain of aspartic acid is protected by t-butyl
  MOD_RES 16: note = The sidechain of lysine is protected by Boc (t-butyloxycarbonyl)
  MOD_RES 19: note = The sidechain of glutamine is protected by a Trt (Trityl) protecting group
  MOD_RES 20: note = Lys at position 20 is chemically modified through conjugation to the epsilon-amino group of the K side-chain with (2-[2-(2-Amino-ethoxy)-ethoxy]-acetyl)2-(gamma-Glu(t- butyl))-CO-(CH2)18-CO2-(t-butyl).
  MOD_RES 24: note = The sidechain of glutamine is protected by a Trt (Trityl) protecting group
  MOD_RES 25: note = The sidechain of Tryptophan is protected by Boc (t-butyloxycarbonyl)
  MOD_RES 32: note = The sidechain of serine is protected by t-butyl
  MOD_RES 33: note = The sidechain of serine is protected by t-butyl
  MOD_RES 39: note = The sidechain of serine is protected by t-butyl
  MOD_RES 39: note = C-terminal amino acid is amidated as a C-terminal primary amide

SEQ ID NO: 5 - protein, 39 aa, synthetic construct; identical to SEQ ID NO: 1
  SITE 2: note = X at position 2 is Aib
  SITE 13: note = X at position 13 is Aib
  MOD_RES 20: note = Lys at position 20 is chemically modified through conjugation to the epsilon-amino group of the K side-chain with (2-[2-(2-Amino-ethoxy)-ethoxy]-acetyl)2-(gamma-Glu)-C O-(CH2)18-CO2H
  MOD_RES 39: note = C-terminal amino acid is amidated as a C-terminal primary amide

SEQ ID NO: 6 - protein, 39 aa, synthetic construct; identical to SEQ ID NO: 1
  MOD_RES 1: note = N-terminus is protected by Boc (t-butyloxycarbonyl)
  MOD_RES 1: note = The sidechain of tyrosine is protected by t-butyl
  SITE 2: note = X at position 2 is Aib
  MOD_RES 3: note = The sidechain of glutamic acid is protected by t-butyl
  MOD_RES 5: note = The sidechain of threonine is protected by t-butyl
  MOD_RES 7: note = The sidechain of threonine is protected by t-butyl
  MOD_RES 8: note = The sidechain of serine is protected by t-butyl
  MOD_RES 9: note = The sidechain of aspartic acid is protected by t-butyl
  MOD_RES 10: note = The sidechain of tyrosine is protected by t-butyl
  MOD_RES 11: note = The sidechain of serine is protected by t-butyl
  SITE 13: note = X at position 13 is Aib
  MOD_RES 15: note = The sidechain of aspartic acid is protected by t-butyl
  MOD_RES 16: note = The sidechain of lysine is protected by Boc (t-butyloxycarbonyl)
  MOD_RES 19: note = The sidechain of glutamine is protected by a Trt (Trityl) protecting group
  MOD_RES 20: note = Lys at position 20 is chemically modified through conjugation to the epsilon-amino group of the K side-chain with (2-[2-(2-Amino-ethoxy)-ethoxy]-acetyl)2-(gamma-Glu(t- butyl))-CO-(CH2)18-CO2-(t-butyl).
  MOD_RES 24: note = The sidechain of glutamine is protected by a Trt (Trityl) protecting group
  MOD_RES 25: note = The sidechain of Tryptophan is protected by Boc (t-butyloxycarbonyl)
  MOD_RES 32: note = The sidechain of serine is protected by t-butyl
  MOD_RES 33: note = The sidechain of serine is protected by t-butyl
  MOD_RES 39: note = The sidechain of serine is protected by t-butyl
  MOD_RES 39: note = C-terminal amino acid is amine is attached to Resin

SEQ ID NO: 7 - protein, 10 aa, synthetic construct
  MOD_RES 3: note = The sidechain of serine is protected by t-butyl
  MOD_RES 4: note = The sidechain of serine is protected by t-butyl
  MOD_RES 10: note = The sidechain of serine is protected by t-butyl
  MOD_RES 10: note = C-terminal amino acid is amidated as a C-terminal primary amide
GPSSGAPPPS

SEQ ID NO: 8 - protein, 8 aa, synthetic construct
  MOD_RES 1: note = N-terminus is protected by Fmoc
  MOD_RES 3: note = The sidechain of glutamine is protected by a Trt (Trityl) protecting group
  MOD_RES 4: note = The sidechain of Tryptophan is protected by Boc (t-butyloxycarbonyl)
FVQWLIAG

SEQ ID NO: 9 - protein, 7 aa, synthetic construct
  MOD_RES 1: note = N-terminus is protected by Fmoc.
  MOD_RES 1: note = The sidechain of aspartic acid is protected by t-butyl
  MOD_RES 2: note = The sidechain of lysine is protected by Boc (t-butyloxycarbonyl)
  MOD_RES 6: note = Lys at position 6 is chemically modified through conjugation to the epsilon-amino group of the K side-chain with (2-[2-(2-Amino-ethoxy)-ethoxy]-acetyl)2-(gamma-Glu(t- butyl))-CO-(CH2)18-CO2-(t-butyl).
DKIAQKA

SEQ ID NO: 10 - protein, 14 aa, synthetic construct
  MOD_RES 1: note = N-terminus is protected by Boc (t-butyloxycarbonyl)
  MOD_RES 1: note = The sidechain of tyrosine is protected by t-butyl
  SITE 2: note = X at position 2 is Aib
  MOD_RES 3: note = The sidechain of glutamic acid is protected by t-butyl
  MOD_RES 5: note = The sidechain of threonine is protected by t-butyl
  MOD_RES 7: note = The sidechain of threonine is protected by t-butyl
  MOD_RES 8: note = The sidechain of serine is protected by t-butyl
  MOD_RES 9: note = The sidechain of aspartic acid is protected by t-butyl
  MOD_RES 10: note = The sidechain of tyrosine is protected by t-butyl
  MOD_RES 11: note = The sidechain of serine is protected by t-butyl
  SITE 13: note = X at position 13 is Aib
YXEGTFTSDYSIXL

(19) United States
(12) Patent Application Publication
(10) Pub. No.: US 2025/0304642 A1
(43) Pub. Date: Oct. 2, 2025

(54) PROCESS FOR PREPARING A GIP/GLP1 DUAL AGONIST

(71) Applicant: ELI LILLY AND COMPANY, Indianapolis, IN (US)

(72) Inventors: Michael E. KOBIERSKI, Greenwood, IN (US); Michael E. KOPACH, Greenwood, IN (US)

(21) Appl. No.: 19/233,974

(22) Filed: Jun. 10, 2025

Related U.S. Application Data
(63) Continuation of application No. 18/800,392, filed on Aug. 12, 2024.
Application No. 18/800,392 is a continuation of application No. PCT/US23/80440, filed on Nov. 20, 2023.
(60) Provisional application No. 63/426,904, filed on Nov. 21, 2022.
(60) Provisional application No. 63/430,430, filed on Dec. 6, 2022.

(51) Int. Cl. C07K 14/575

(52) U.S. Cl. CPC C07K 14/575

(57) ABSTRACT

The present invention provides novel intermediates and processes useful in the manufacture of tirzepatide, or a pharmaceutically acceptable salt thereof.

The present invention provides improved processes and intermediates for making a GIP/GLP1 dual agonist peptide, tirzepatide, or a pharmaceutically acceptable salt thereof.

Diabetes mellitus is a chronic disorder characterized by hyperglycemia resulting from defects in insulin secretion, insulin action, or both. In type 2 diabetes mellitus (“T2D”), the combined effects of impaired insulin secretion and insulin resistance are associated with elevated blood glucose levels. The GIP/GLPI dual agonist, tirzepatide is described and claimed in U.S. Pat. No. 9,474,780 (“780 Patent”). Tirzepatide can be useful in the treatment of T2D.

U.S. Pat. No. 9,474,780 generally describes peptides and a method for making a GIP/GLP1 dual agonist. WO2020/159949 describes methods of making tirzepatide; however, there is a need for linear process improvements to facilitate large scale commercial production.

There is a need for processes and intermediates to enable improved technology for production of tirzepatide having a combination of advantages including commercially desired purity and efficiency. Similarly, there is a need for efficient and environmentally “green” processes, including stable intermediates to provide tirzepatide with fewer purification steps. Improved technology is also needed to provide tirzepatide manufacturing processes producing minimal waste streams for both environmental and operator enhanced safety. The preparation of large-scale, pharmaceutically-elegant tirzepatide presents a number of technical challenges that may affect the overall yield and purity. There is a need for processes to avoid the use of transition metals and/or harsh reaction conditions that are incompatible with peptide synthesis.

The present invention seeks to meet these needs by providing novel intermediates and processes useful in the manufacture of tirzepatide (SEQ ID NO:5), or a pharmaceutically acceptable salt thereof. The improved tirzepatide manufacturing processes of the present invention provide intermediates and process reactions embodying a combination of advances, including an efficient route having fewer steps, while at the same time maintaining high quality and purity. Importantly, the improved processes and intermediates decrease resource intensity and minimize waste streams.

The improved processes described herein provide various embodiments of intermdiates useful for production of tirzepitide.

The present invention provides a compound of SEQ ID NO:1, or a pharmaceutically acceptable salt thereof. The present invention provides a compound of SEQ ID NO:2, or a pharmaceutically acceptable salt thereof. The present invention provides a compound of SEQ ID NO:3, or a pharmaceutically acceptable salt thereof.

The present invention provides a process to prepare tirzepatide, comprising preparing the protected compound, or pharmaceutically acceptable salt, of a compound of SEQ ID NO:1. The present invention provides a process to prepare tirzepatide, comprising deprotecting a compound, or pharmaceutically acceptable salt, of a compound of SEQ ID NO:1. The present invention provides a process to prepare tirzepatide, comprising cleaving a compound from a resin, or pharmaceutically acceptable salt, of a compound of SEQ ID NO:2. The present invention provides a process to prepare tirzepatide, comprising deprotecting a compound, or pharmaceutically acceptable salt, of a compound of SEQ ID NO:3. The present invention provides a process to prepare tirzepatide (SEQ ID NO:5), comprising acylating a lysine to form a compound, or pharmaceutically acceptable salt, of a compound of SEQ ID NO:4 and then deprotecting to form tirzepatide.

Provided is a process to prepare the mtt protected epsilon amino group of a lysine amino acid of a resin bound protected peptide of SEQ ID NO:1. Provided is a process to selectively deprotect a mtt protected epsilon amino group of a lysine amino acid of a resin bound protected peptide of SEQ ID NO:1 to form a protected peptide of SEQ ID NO:2. Provided is a process to selectively cleave the resin bound protected peptide of SEQ ID NO:2 to form the the compound of SEQ ID NO:3. Provided is a process to acylate an unprotected epsilon amino group of a lysine of SEQ ID NO:3 with Compound 1 to form the compound of SEQ ID NO:4.

Provided is a process for determining the chiral purity for the preparation of a peptide by SPPS by using a chiral high-performance liquid chromatography-electrospray ionization mass spectrometry (HPLC-ESI-MS/MS) method.

In an embodiment is a process to prepare tirzepatide (SEQ ID NO:5) comprising
a) deprotecting a compound of SEQ ID NO:1 to provide a compound of SEQ ID NO:2;
b) remove resin to provide a compound SEQ ID NO:3;
c) couple SEQ ID NO:3 with Compound 1 to provide SEQ ID NO:4;
d) deprotect SEQ ID NO:4 to provide tirzepatide (SEQ ID NO:5).

In an embodiment is a process to prepare tirzepatide (SEQ ID NO:5) comprising
a) deprotecting a compound of SEQ ID NO:1 to provide a compound of SEQ ID NO:2;
b) couple SEQ ID NO:2 with Compound 1 to provide SEQ ID NO:6;
c) deprotect and remove resin to yield tirzepatide (SEQ ID NO:5),
d) remove SEQ ID NO:6 from resin to provide SEQ ID NO:4; and
e) deprotect SEQ ID NO:4 to yield tirzepatide (SEQ ID NO:5).

In an embodiment is a process to prepare tirzepatide (SEQ ID NO:5) comprising
a) deprotecting a compound of SEQ ID NO:1 to provide a compound of SEQ ID NO:2;
b) couple SEQ ID NO:2 with Compound 1 to provide SEQ ID NO:6;
c) remove SEQ ID NO:6 from resin to provide SEQ ID NO:4; and
d) deprotect SEQ ID NO:4 to yield tirzepatide (SEQ ID NO:5).

In an embodiment is a continuous flow process for preparing tirzepatide (SEQ ID NO:5) comprising:
a) deprotecting a compound of SEQ ID NO:1 to provide a compound of SEQ ID NO:2;
b) remove resin to provide a compound SEQ ID NO:3; wherein nanofiltration is optionally place in line to purify and provide solvent exchange;
c) couple SEQ ID NO:3 with Compound 1 to provide SEQ ID NO:4; wherein nanofiltration is placed in line to purify and provide solvent exchange.
d) deprotect SEQ ID NO:4 to provide tirzepatide (SEQ ID NO:5), wherein nanofiltration is placed in line to purify and provide solvent exchange.

In an embodiment is a continuous flow process for preparing tirzepatide (SEQ ID NO:5) comprising:
a) deprotecting a compound of SEQ ID NO:1 to provide a compound of SEQ ID NO:2;
b) couple SEQ ID NO:2 with Compound 1 to provide SEQ ID NO:6;
c) remove SEQ ID NO:6 from resin to provide SEQ ID NO:4; and
d) deprotect SEQ ID NO:4 to yield tirzepatide (SEQ ID NO:5), wherein nanofiltration is placed in line to purify and provide solvent exchange.

In an embodiment to improve efficiency continuous flow process and chromatographic purification for preparing tirzepatide (SEQ ID NO:5) comprising:
a) deprotecting a compound of SEQ ID NO:1 to provide a compound of SEQ ID NO:2;
b) remove resin to provide a compound SEQ ID NO:3; wherein chromatography and nanofiltration are optionally placed in line to purify and provide solvent exchange.
c) couple SEQ ID NO:3 with Compound 1 to provide SEQ ID NO:4; wherein nanofiltration is placed in line to purify and provide solvent exchange.
d) deprotect SEQ ID NO:4 to provide tirzepatide (SEQ ID NO:5), wherein chromatography nanofiltration is placed in line to purify and provide solvent exchange.

In an embodiment is a continuous flow process and chromatographic purification for preparing tirzepatide (SEQ ID NO:5) comprising:
a) deprotecting a compound of SEQ ID NO:1 to provide a compound of SEQ ID NO:2;
b) remove resin to provide a compound SEQ ID NO:3; wherein chromatography and nanofiltration are placed in line to purify and provide solvent exchange.
c) couple SEQ ID NO:3 with Compound 1 to provide SEQ ID NO:4; wherein nanofiltration is placed in line to purify and provide solvent exchange.
d) deprotect SEQ ID NO:4 to provide tirzepatide (SEQ ID NO:5), wherein chromatography nanofiltration is placed in line to purify and provide solvent exchange.

Provided is a process to selectively acylate an unprotected lysine amino acid. Provided is a process to selectively acylate a lysine amino acid in a peptide comprising coupling a resin bound peptide-Lysine-NH₂ with t-butyl-eicosanedioyl-Glu-(O-tert-butyl)-(8-amino-3,6-dioxaoctanoic acid)-(8-amino-3,6-dioxaoctanoic acid)-OH. Provided is a process to prepare tirzepatide, comprising deprotecting a compound of SEQ ID NO:4, or a pharmaceutically acceptable salt thereof.

The present invention provides a process to prepare tirzepatide, comprising cleaving a compound from a resin, or pharmaceutically acceptable salt, of a compound of SEQ ID NO:2. The present invention provides a process to prepare tirzepatide, comprising deprotecting a compound, or pharmaceutically acceptable salt, of a compound of SEQ ID NO:3. The present invention provides a process to prepare tirzepatide, comprising acylating a lysine to form a compound, or pharmaceutically acceptable salt, of a compound of SEQ ID NO:4.

Provided is a process to selectively acylate a lysine amino acid wherein the resin bound peptide-Lysine-NH2 is a compound of the SEQ ID NO:2:

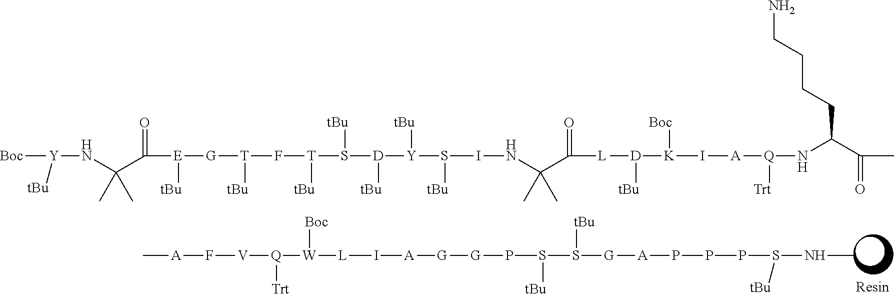

or a pharmaceutically acceptable salt thereof.

The improved process using a lysine MTT protected intermediate is othrogonally protected in higher purity than the analogous intermediate used in known processes. Further, the synthesis of tirzepatide using the MTT protected intermediate on resin is orthogonally deprotected at Lysine 20, with the MTT removed, and coupled with (S)-22-(tert-butoxycarbonyl)-45,45-dimethyl-10,19,24,43-tetraoxo-3,6,12,15,44-pentaoxa-9,18,23-triazahexatetracontanoic acid, providing desired purity. Further, the SEQ ID NO: 6 can be cleaved from the resin to form SEQ ID NO:4, and deprotected to provide tirzepatide, enabling continuous flow synthesis.

Both tirzepatide sythesis processes using MTT deliver improved purity and yield relative to the known linear process. Further, processes herein have the further benefit of potentially being able to reduce the (S)-22-(tert-butoxycarbonyl)-45,45-dimethyl-10,19,24,43-tetraoxo-3,6,12,15,44-pentaoxa-9,18,23-triazahexatetracontanoic acid, stoichiometry to ˜ 1 equivalent due to LPPS conditions, and can be potentially amenable to flow synthesis methods. Reducing the need for excess (S)-22-(tert-butoxycarbonyl)-45,45-dimethyl-10,19,24,43-tetraoxo-3,6,12,15,44-pentaoxa-9,18,23-triazahexatetracontanoic acid can be both environmentally and commercially important for large scale synthesis of peptides.

The determination of chiral purity is critical to the evaluation of the quality of peptide pharmaceutical products. For synthetic peptides, the undesirable D-isomers can be introduced as impurities in amino acid starting materials and can also be formed during peptide synthesis and in some cases during product shelf life. A chiral high-performance liquid chromatography-electrospray ionization tandem mass spectrometry (HPLC-ESI-MS/MS) method is described that facilitates rapid and accurate determination of amino acid chiral purity in a peptide. The peptide is hydrolyzed in deuterated acid to facilitate correction for any racemization occurring during this step of sample preparation, and the amino acids are subsequently separated by chiral chromatography interfaced with ESI-MS/MS for quantitation. The amino acid samples are analyzed directly following hydrolysis by chromatographic separation and extraction of selected ion response, providing efficiency and simplicity by avoiding the derivatization steps required by traditional methodologies. Validation feasibility of the method is described for all nineteen chiral proteogenic amino acids, and the practical application is demonstrated for the analysis of model peptides. The method was proven capable of quantitative determination of trace levels of the D-isomer impurities across the 0.1%- 1.0% range of interest.

As used herein, the following abbreviations have the meanings as set forth herein: “SPPS” means Solid Phase Peptide Synthesis, “MTT” and/or “mtt” means 4-methyltrityl, “Fmoc” means fluorenylmethyloxycarbonyl chloride, “Pip” means piperidine, “DIC” means diisopropylcarbodiimide, “Oxyma” means Ethyl cyanohydroxyiminoacetate, “DCM” means dichloromethane, “IPA” means isopropanol, “MTBE” means methyl-tert-butyl ether, “TFA” means trifluoroacetic acid, “TIPS” means triisopropylsilane, “DTT” means dithiothreitol, “UPLC” means Ultra High Performance Liquid Chromatography, “HFIP” means hexafluoroisopropanol, “CTC” means chlorotrityl, “HATU” means (1-[bis(dimethylamino)methylene]-1H-1,2,3-triazolo[4,5-b]pyridinium 3-oxide hexafluorophosphate, “TFET” means 2,2,2-trifluoroethanethiol, “DIEA” means N,N-diisopropylethylamine, “AEEA” means 17-amino-10-oxo-3,6,12,15 tetraoxa-9-aza heptadecanoic acid, “TCEP” means tris(2-carboxyethyl)phosphine, “DCU” means dicyclyhexylurea, “DCC” means dicyclhexylcarbodiimide, “TMSA” means trimethylsilyalmide, “HOBt” means hydroxybenzotriazole, “HRMS” means high resolution mass spectrometry, “LPPS” means liquid phase peptide synthesis, “MSMPR” means mixed product mixed suspension reactor, “MPA” means mobile phase A, “MPB” means mobile phase B, “L-GSH” means L-glutathione reduced solution, “TZP” means tirzepatide, “AP” means active pharmaceutical, and “API” means active pharmaceutical ingredient, “PyBOP” means (benzotriazol-1-yloxy) tripyrrolidinophosphonium hexafluorophosphate), “DEA” means diethylamine, “TBTU” means 2-(1H-benzotriazole-1-yl)-1,1,3,3-tetramethylaminium tetrafluoroborate, “TNTU” means 2-(5-Norbornene-2,3-dicarboximido)-1,1,3,3-tetramethyluronium Tetrafluoroborates, “PyOxim” means 1-Cyano-2-ethoxy-2-oxoethylideneaminooxy-tris-pyrrolidino-phosphonium hexafluorophosphate, “PyClock” means 6-chloro-benzotriazole-1-yloxy-tris-pyrrolidinophosphonium hexafluorophosphate. As presented herein, amino acid one letter abbreviations are presented in bold print, while atoms are presented as unbolded text, and generally in smaller font, to distinguish from one letter amino acid abbreviations. As used herein, when an amino acid abbreviation appears with a number above the amino acid, the number refers to the corresponding amino acid position in the final tirzepatide product. The numbers are provided for convenience and the appearance or absence of such numbers in a sequence does not influence the amino acid sequence or the peptide indicated in such sequence. As used herein, the term “protected” means that a protecting group is attached to at the indicated position. The artisan will recognize that a variety of protecting groups are well known, and alternative protecting groups may be suitable for a particular process. As used herein “in line” refers to continuous flow synthesis, wherein the stated feature is provided with synthesis, such that the synthesis process may continue without interruption. In an embodiment, in line nanofiltration may be used for purification and/or solvent exchange as desired in the synthesis. In an embodiment, in line chromatography may be used in line during the synthesis process.

The artisan will appreciate that there are alternative resins for building the peptides presented herein. For example, Sieber and Rink amide resins are well known to the artisan for preparing peptides disclosed herein; however, alternative resins may be selected for the preparation of peptides described herein. For example, but not limited to, 2-CTC and related resins may be used to prepare a target peptide, followed by a C terminus amidation step.

Processes described herein are suitable for telescoped steps, for example, multiple process steps running concurrently, encompassing multi-unit operations, enabling the assembly of a number of preassembled peptide units via continuous unit operations, or “continuous flow”. The continuous flow process is enabled by fume hood design supporting continuous flow. Continuous flow synthesis may allow for safe, cost effective, efficient continuous manufacturing to meet commercial demands for peptides.

Processes provided herein may be conducted using nanofiltration for purification and solvent exchange to remove the requirement for additional isolation steps, and to enable a continuous processing platform.

Unit operation based, continuous manufacturing skids, located in fume hoods, may be used to execute the peptide unit coupling reactions in flow. The feeds for the peptide unit coupling reactions are precisely controlled to achieve high reaction conversion while maintaining tight stoichiometric control, irrespective of expected variability from the solid phase synthesized fragments. Feed flow rates for continuous flow processes may be automatically updated as the process requires.

Nanofiltration may be implemented for the purification and solvent exchange of the coupled peptide solutions. Nanofiltration may eliminate an isolation step for an amorphous solid, and may enable a fully telescoped process through the peptide unit reactions. A standby nanofiltration skid may allow for continued manufacturing during periodic maintenance of a nanofiltration skid.

Peptide solutions may be precipitated and isolated in a dual train, batch precipitation and isolation. Once the crude peptide is assembled, the resulting peptide solution may be precipitated and isolated using batch precipitation and isolation equipment. The duals trains may enable continuous flow reactions to continue as the batch precipitation and isolation unit operations are executed. Pack-off barriers may be used to encompass o-ring canister packaging technology to protect both the operator and the facility.

Equipment used in the synthetic peptide manufacturing process may be equipped with clean-in-place circuits and cleaning batch recipes to ensure cleaning is expedient, efficient, and effective.

Solid phase synthesis may allow for the sequestration of the growing peptide fragment, alleviating the need for amorphous intermediate isolations and the requisite byproduct purge and washing steps that may be required to enable a sequential build. In traditional liquid phase coupling, complex isolation steps are generally required for such a purge of byproduct. To eliminate the complexity of intermediate isolations between individual unit couplings, a washing of coupled peptide while in the solution phase may be used.

Nanofiltration can provide a means of concentrating, washing, and exchanging solvents following a coupling process. Due to the challenging polar aprotic solvents required for peptide solubility, non-traditional ceramic membranes may provide a useful alternative to more typical polymeric membranes.

Nanofiltration can provide an efficient, scalable, and cost-effective method of integrating multiple continuous coupling steps in a full peptide assembly system. Nanofiltration technology enables use of a continuous synthesis platform as it removes the need for intermediate isolations during the process steps which would be time consuming. Nanofiltration can be effective for peptides of a wide range of molecular weights, allowing for the platform to be applicable to multiple peptide projects.

Ceramic membranes are made from inorganic materials such as, but not limited to alumina, zirconia oxides, and silicon carbide. The inorganic materials used to make ceramic membranes allow them to be used with aggressive media, e.g., acids and strong solvents, where polymeric membranes might fail making them suitable for use in the liquid phase coupling reactions described herein. Ceramic membranes can be made in a tubular cross flow configuration suitable for use with the continuous coupling steps described herein. Ceramic membranes are available from various suppliers including, but not limited to, Altech (Gladbeck, Germany); CoorsTek (Golden, Colorado); Inopor (Scheßlitz, Germany); Mantec Technical Ceramics Ltd (Staffordshire, England); and Orelis Environnement/Alsys group (Salindres, France).

Methodology of underivatized amino acids may be used with stationary phases and optimized for the determination of the chiral purity through trace level quantitation of D-isomers in synthetic peptides. Samples were hydrolyzed in deuterium chloride (DC1) so that amino acids racemized during the process of the sample preparation can be excluded by Mass Spectrographic detector. The elimination of sample derivatization from the process may facilitate a simplified method with increased throughput potential. Data are presented to demonstrate the chiral HPLC-ESI-MS/MS analysis of mixtures of all 19 chiral D/L natural amino acid pairs with quantitation of the D-isomer achieved by monitoring the extracted ion chromatogram (EIC) associated with a peptide ion specific to each amino acid. The feasibility of the method for quantitation of trace levels of D-isomers in L-isomer matrices is demonstrated through the determination of linearity, accuracy, precision, and limit of detection/limit of quantitation to support a synthetic peptide manufacturing process control limit of 0.5% and a method reporting limit of 0.1% for each D-isomer in alignment with pharmaceutical industry regulatory guidances for synthetic peptides. Chiral HPLC-ESI-MS/MS method was validated through application to the analysis of four model peptides, each consisting of 8-14 amino acid residues.

The four model peptides used in this study consist of 8-14 amino acid residues each and are described by amino acid content with number of residues in Tables 1. L-and D-isomer peptide materials were synthesized as amorphous solids at high purity. The peptides were fully characterized for identity, purity, and content. The samples were synthesized as “protected” peptides, where the active side chains of amino acids (such as hydroxyl, carboxyl, amino, etc.) are protected with functional groups such as Fmoc, trityl (Trt), tert-butyloxycarbonyl (Boc), and tertiary butyl (tBu), as listed in Table 1.Chiral purity analyses of the peptide samples were also executed using the traditional GCMS and LCMS methodologies for comparison.

TABLE 1
The amino acid content of the model peptide samples

| Peptide A | Peptide B | Peptide C | Peptide D |
|---|---|---|---|
| (10 aa's) | (8 aa's) | (8 aa's) | (14 aa's) |
| Ala | Ala | Ala × 2 | Aib* × 2 |
| Gly × 2 | Gly | Fmoc-Asp(tBu) | Asp(OtBu) |
| Pro × 4 | Gln(Trt) | Glu(tBu) | Glu(OtBu) |
| Ser(tBu) × 3 | Ile | Gln(Trt) | Gly |
| | Leu | Ile | Ile |
| | Fmoc-Phe | Lys(Boc) × 2 | Leu |
| | Trp(Boc) | | Phe |
| | Val | | Ser(tBu) × 2 |
| | | | Thr(tBu) × 2 |
| | | | Tyr(tBu) × 2 |

*Aib (2-aminoisobutyric acid) is a non-chiral non-proteogenic amino acid

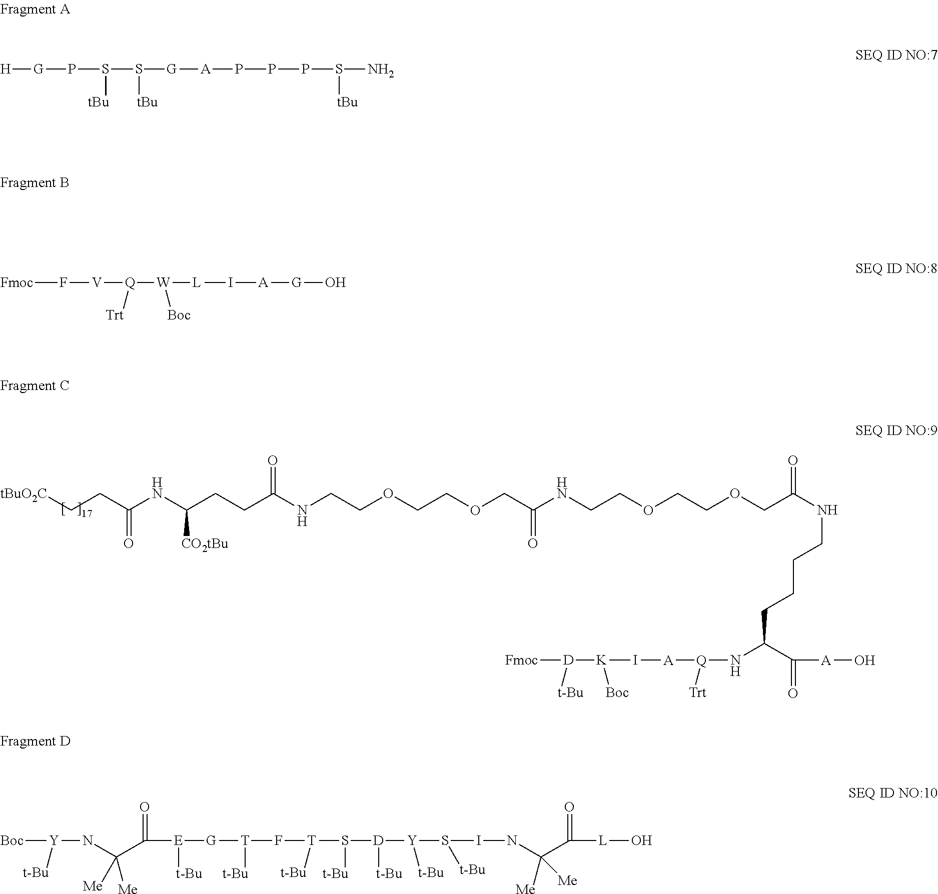

### Sample Preparation

The nominal target concentration for each amino acid was 0.1 mg/mL. The amino acid standards were prepared in water, with the exceptions of aspartic acid, cysteine, and tyrosine which required approximately 4% formic acid for solubility (these three amino acids were dissolved in formic acid first, then diluted to volume with water). Amino acid standard solutions were prepared at concentrations of approximately 0.0005 mg/mL-0.002 mg/mL to represent a range of 0.05-2.0% of amino acid nominal concentrations in a peptide hydrolysis mixture.

For the analysis of the Fmoc-protected peptide Fragments B and C, the samples were incubated at concentrations of approximately 1 mg/mL (to provide amino acid concentrations of at minimum 0.1 mg/mL for a peptide consisting of 10 amino acids) in a piperidine solution (3 mL piperidine+10 mL dichloromethane) for 15 min at 2-8° C. to remove the Fmoc-protecting group prior to hydrolysis. Following Fmoc removal, the samples were dried for 4 hours under a nitrogen stream. All dried peptide samples were digested by acid hydrolysis in 1.0 mL volumes of 5N DCI at a concentration of approximately 1 mg/mL in 10 mL glass vials with Teflon heat-resistant caps at 110° C. for 4 hours. To prevent the oxidation of tryptophan to N′-formylkynurenine or related species, samples of Peptide Fragment B were mixed with 5N DCI+1% phenol, purged with nitrogen, and then carefully sealed with Teflon tape and parafilm around the vial caps prior to hydrolysis at 110° C. In addition to the cleavage of the peptide into its individual amino acid constituents, the remaining protecting groups (tBu, Boc, and Trt) are removed during the hydrolysis step. Following hydrolysis, the samples were cooled to ambient temperature and 2 mL of water was added. The samples were then dried for 12 hours using a Genevac™ EZ-2 Elite HCl compatible centrifuge vacuum concentrator (Genevac Ltd. Ipswitch, UK) to remove the acid by lyophilization. As a final step, the samples were reconstituted to the original 1 mL volume in water prior to analysis by chiral HPLC-ESI-MS/MS. Samples for proline analysis using the Chiralpak® ZWIX(−) column method were further diluted 1:1 with water prior to injection.

### Chiral HPLC-ESI-MS/MS

The UPLC system included a binary solvent manager and an autosampler. Separations were carried out on two Crownpak®CR-I(+) 150×3.0 mm I.D. 5 μm columns (Chiral Technologies, Inc. West Chester, PA) connected in series using isocratic elution with a mobile phase composition of 5% A (0.5% TFA in water) and 95% B (85 ACN/15 EtOH/0.5 TFA; v/v/v). The flow rate was 0.4 mL/min, injection volume was 1 μL, and column temperature was 30° C. Separations were also performed using a single Chiralpak® ZWIX(−) 150×3.0 mm I.D. 5 μm column (Chiral Technologies, Inc. West Chester, PA), and the mobile phase used with this column was isocratic and consisted of 25 mM formic acid, 25 mM ammonium formate in methanol/water (98/2 v/v).

The UPLC was coupled to a mass spectrometer with Turbo Spray IonDrive operated in Multiple Reaction Monitoring (MRM) mode. The separation employed chromatography conditions described above with a divert valve programmed to divert the first two minutes to waste. The mass spectrometer was operated in positive ion polarity with ionspray voltage set to 5500 V and temperature set to 550° C. The MRM transitions for each amino acid can be found in Table 2 with respective Q1 and Q3 m/z values (both set to unit resolution) and as well as optimized declustering potential, entrance potential, collision energy, and collision cell exit potential values. The 132.1 to 86.0 m/z transition represents both Leu and Ile as both amino acids peptide in this manner, this transition not providing any specificity between this pair of isobaric amino acids. However, the chromatography is able to effectively separate the Leu and Ile isomers (20, 25), and in addition the 132.1 to 69.0 m/z transition is specific to isoleucine, providing a transition that can be used for isoleucine quantitation. Other relevant instrument parameters included curtain gas of 20 PSI, ion source gas 1 of 40 PSI, ion source gas 2 of 60 PSI, and collision gas set to low. Data acquisition and processing were performed using Analyst software (SCIEX).

TABLE 2
List of targeted amino acids with the associated MRM conditions.

| | Q1 | Q3 | Dwell | DP | EP | CE | CXP |
|---|---|---|---|---|---|---|---|
| Amino Acid | (m/z) | (m/z) | (ms) | (V) | (V) | (V) | (V) |
| Glycine | 76.1 | 30.0 | 10 | 6 | 7.6 | 19 | 14 |
| Alanine | 90.1 | 44.1 | 10 | 6 | 4.5 | 17 | 6 |
| Serine | 106.1 | 60.0 | 10 | 6 | 10.5 | 15.5 | 7 |
| Proline | 116.1 | 70.0 | 10 | 20 | 13.5 | 21 | 10 |
| Valine | 118.1 | 55.0 | 10 | 11 | 13.5 | 27 | 8 |
| Threonine | 120.1 | 74.0 | 10 | 105 | 14.5 | 25 | 7 |
| Cysteine | 122.0 | 59.0 | 10 | 20 | 14 | 19 | 10 |
| Leucine/ | 132.1 | 86.1 | 10 | 8 | 14.5 | 13 | 10 |
| isoleucine | | | | | | | |
| Isoleucine 2 | 132.1 | 69.0 | 10 | 8 | 14.5 | 23 | 8 |
| Aspartic Acid/ | 134.1 | 74.1 | 10 | 7 | 14.5 | 19 | 10 |
| Asparagine | | | | | | | |
| Lysine | 147.1 | 84.0 | 10 | 15 | 13.5 | 23 | 10 |
| Glutamic Acid/ | 148.1 | 84.0 | 10 | 21 | 14.5 | 21 | 10 |
| Glutamine | | | | | | | |
| Methionine | 150.2 | 104.0 | 10 | 6 | 12 | 15 | 12 |
| Histidine | 156.1 | 110.0 | 10 | 16 | 13 | 19 | 12 |
| Phenylalanine | 166.1 | 103.0 | 10 | 11 | 14 | 37 | 12 |
| Arginine | 175.2 | 70.1 | 10 | 40 | 11 | 27 | 8 |
| Tyrosine | 182.1 | 165.2 | 10 | 20 | 11 | 13 | 8 |
| Tryptophan | 205.1 | 188.0 | 10 | 20 | 14 | 19 | 10 |

### Chiral HPLC Separation of Amino Acids

Both crown ethers and quinine-/quinidine-derived zwitterionic stationary phases used in hydrophilic interaction chromatography (HILIC) mode have proved to be highly effective chiral selectors for the separation of amino acid enantiomers. The crown ether of the Crownpak® stationary phase strongly interacts with amino groups that are protonated by the highly acidic TFA mobile phase, then the binapthyl moiety facilitates stereospecific retention due to the structural difference of the amino acid entantiomers. The crown ether provides separation of all D/L amino acid pairs with the exception of D/L proline due to poor affinity with the amino group of the secondary amine. The proline chiral separation instead is provided by Chiralpak® ZWIX, where this alkaloid-derived zwitterionic stationary phase is assumed to induce selectivity through ion pairing in a mobile phase containing both acidic and basic additives. The elution order of the separation of isomeric analytes provided by both of these packings can be adjusted by selecting the appropriate chiral configuration of the stationary phase. The Crownpak®CR-I(+) and Chiralpak® ZWIX(−) packings were specifically chosen for their ability to facilitate elution of the D-isomer prior to the L-isomer, thereby minimizing any interference due to tailing of the L-isomer peak which is present at high concentration.

The chiral chromatography methods using the Crownpak®CR-I(+) and Chiralpak® ZWIX(−) columns were designed by referencing parameters previously described for the analysis of D-amino acids in foods. However, in the case of the current work two Crownpak®CR-I (+) columns were connected in series to provide optimal resolution. For determination of the D-isomer impurities in synthetic peptides, the chromatography separations were combined with a high-low sample concentration chromatography strategy (26). High-low chromatography derives its name from the use of a pair of sample solutions, the “high” corresponding to a nominal sample concentration of approximately 0.1 mg/mL for each individual L-amino acid in the hydrolysis solution mixture and the “low” representing a sample concentration of approximately 0.001 mg/mL. Calculations of D-isomer % were based on comparisons of the chromatograms obtained from the two solutions, where the peak area of the L-isomer present in the low concentration sample effectively serves as an external standard calibrator for % area quantitation of the D-isomer peak areas measured in the high concentration sample:

$$\% D = \frac{{Area}_{D}}{100 \times {Area}_{L}} \times 100$$

Where Area_{D} represents the peak area of the D-isomer in the 0.1 mg/ml sample and AreaL represents the peak area of the L-isomer in the 0.001 mg/ml sample. The significant advantages of this strategy are two-fold: sensitivity amplification facilitating reduced detection limits and simplified calibration for quantitation. The high/low strategy eliminates the need for a set of 19 external standards corresponding to each amino acid that would otherwise be used.

Chiral HPLC-ESI-MS/MS Analysis of Amino Acids

In the chiral HPLC-ESI-MS/MS analyses, protonated molecular ions ([M+H]⁺) are selected as the precursor ions of all targeted compounds based on the expected m/z value corresponding to each amino acid non-racemized (i.e. non-deuterated) in DC1. Peptideation is induced and the ion signal responses of the product ions specific to each amino acid are measured. The precursor ions and product ions used for quantitation of each amino acid are listed in Table 5. Following sample preparation and analysis, the extracted ion chromatogram (EIC) associated with the response of the product ion associated with each amino acid will represent the D-and L-amino acids intrinsically present in the peptide prior to hydrolysis. As described in the Introduction, any amino acids racemized during hydrolysis are excluded as precursor ions because of an increase in molecular mass of one atomic mass unit through incorporation of deuterium. Using this approach, all amino acids present in a peptide can be analyzed post-hydrolysis in a single chiral HPLC-ESI-MS/MS experiment by monitoring the EICs corresponding to the expected product ions eluting at the specified chromatographic retention times. To confirm sensitivity, linearity, and accuracy to support a method reporting limit of 0.1% and a quantitation range of 0.1%-1.0% D-isomer, a set of standards was prepared at six levels (0.05%, 0.1%, 0.2%, 0.5%, 1.0%, and 2.0%) and analyzed for each of the amino acids. For all amino acids, the detector response demonstrated excellent sensitivity with response at 0.1% generating a S/N>10 and linearity with R²=0.9900 (see Table 3). D-isomer spikes were also prepared into the corresponding L-isomer matrices. Spiked samples were introduced at levels of 0.1%, 0.2%, 0.5%, and 1.0% into the corresponding L-isomer matrices at the nominal concentration of 0.1 mg/mL. The capability of the method to determine the D-isomer responses with an accuracy of approximately 80-120% recovery was demonstrated, where % recovery was based on the weighed spiked sample preparations and spike response compared directly to the corresponding standard curve. The sensitivity, linearity, and accuracy data representing all 19 chiral amino acids are listed in Table 3, and the results support the quantitative capabilities of the method.

TABLE 3
The chiral HPLC-ESI-MS/MS sensitivity, linearity, and accuracy
data representing the quantitation of D-isomers of all 19
natural chiral amino acids supporting a range of 0.10%-1.0%.

| | S/N at | Linearity | Accuracy |
|---|---|---|---|
| | 0.10% | (R² Across | (% Recovery Across |
| Amino Acid | Level | Range of 0.05-2.0%) | Range of 0.1-1.0%) |
| Alanine | 16 | 0.9957 | 88-106 |
| Serine | 55 | 0.9996 | 92-118 |
| Proline | 34 | 0.9995 | 85-116 |
| Valine | 66 | 0.9976 | 99-107 |
| Threonine | 10 | 0.9998 | 82-105 |
| Cysteine | 80 | 0.9991 | 95-102 |
| Leucine | 21 | 0.9970 | 92-109 |
| Isoleucine | 78 | 0.9968 | 96-103 |
| Aspartic Acid/ | 89 | 0.9995 | 95-113 |
| Asparagine | | | |
| Lysine | 409 | 0.9990 | 89-105 |
| Glutamic Acid/ | 90 | 0.9968 | 95-105 |
| Glutamine | | | |
| Methionine | 93 | 0.9995 | 93-115 |
| Histidine | 413 | 0.9999 | 96-105 |
| Phenylalanine | 335 | 0.9968 | 79-108 |
| Arginine | 3570 | 1.0000 | 88-108 |
| Tyrosine | 198 | 1.0000 | 82-106 |
| Tryptophan | 365 | 0.9989 | 93-108 |

Batches of all four model peptides were analyzed and reported by both the chiral HPLC-ESI-MS/MS method and the traditional methodologies for comparison and confirmation. GC-MS with derivatization was utilized for comparative testing of Peptides A and D, while HPLC-MS with derivatization was employed for comparative testing of Peptides B and C. The results are displayed in Table 4 and demonstrate the expected low levels of D-isomers with good agreement between the new and traditional techniques within the inherent variability estimated based on the accuracy and precision associated with each method (approximately +/−0.1%, base on 3×SD for the current method at the 0.1-0.2% D-isomer range and the validation of the GC-MS method (30)).

TABLE 4

| Peptide Sample/ | Chiral HPLC-ESI- | Traditional |
|---|---|---|
| Amino Acid | MS/MS % D-isomer | Methodology Results |
| | | GC-MS (12) |
| | | % D-isomer¹ |
| Peptide A Pro | <0.10 | <0.10 |
| Peptide A Ser | <0.10 | 0.23 |
| Peptide A Ala | 0.11 | <0.10 |
| | | HPLC-MS (5) |
| | | % D-isomer² |
| Peptide B Phe | <0.10 | <0.10 |
| Peptide B Val | <0.10 | <0.10 |
| Peptide B Gln/Glu | <0.10 | 0.19 |
| Peptide B Trp | <0.10 | 0.11 |
| Peptide B Leu | <0.10 | <0.10 |
| Peptide B Ile | <0.10 | <0.10 |
| Peptide B Ala | 0.12 | 0.12 |
| | | HPLC-MS (5) |
| | | % D-isomer² |
| Peptide C Asp | 0.12 | <0.10 |
| Peptide C Lys | 0.14 | <0.10 |
| Peptide C Ile | <0.10 | <0.10 |
| Peptide C Ala | <0.10 | 0.11 |
| Peptide C Gln/Glu | 0.14 | 0.15 |
| | | GC-MS (12) |
| | | % D-isomer¹ |
| Peptide D Tyr | 0.15 | <0.10 |
| Peptide D Glu | 0.13 | 0.20 |
| Peptide D Thr | <0.10 | <0.10 |
| Peptide D Phe | <0.10 | 0.12 |
| Peptide D Ser | 0.14 | <0.10 |
| Peptide D Asp | 0.19 | <0.10 |
| Peptide D Ile | <0.10 | <0.10 |
| Peptide D Leu | <0.10 | 0.15 |

¹Testing of Peptides A and D was performed substantially as described by R. Liardon, S. Ledermann, and U. Ott, Determination of D-amino acids by deuterium labelling and selected ion monitoring, J. Chromatogr. A 203 (1981) 385-395.
²Testing of Peptides B and C was performed substantially as described by K. Kalikova, T. Slechtova, and E. Tesarova, Enantiomeric ratio of amino acids as a tool for determination of aging and disease diagnostics by chromatographic measurement, Separations 3 (2016) 30-48.

The Solid Phase Peptide Synthesis (SPPS) builds are accomplished using standard fluorenylmethyloxycarbonyl chloride (Fmoc) peptide chemistry techniques employing sequential couplings with an automated peptide synthesizer. The resin is swelled with DMF then de-protected using 20% piperidine (Pip)/DMF (3×30 min). Subsequent Fmoc de-protections use 20% Pip/DMF 3×30 min treatments and 4×30 min treatments are used for more difficult couplings. After deprotection, the resin is washed with 5×2 min, 10 volume DMF washes. Amino acid pre-activation uses diisopropylcarbodiimide (DIC)/ethyl cyanohydroxyiminoacetate (Oxyma) DMF solutions at room temp for 30 min. Coupling of the activated amino acid to the resin bound peptide occurs for a specified time for each individual amino acid. Solvent washing with 5×2 min 10 volumes DMF is performed after each coupling. For isolation of the final product, the resin bound product is washed 5×2 min with 10 volume DCM to remove DMF. The resin is washed with 2×2 min 10 volume IPA to remove DCM, washed 5×2 min 10 volume methyl-tert-butyl ether (MTBE), then the product is dried at 40° C. under vacuum. The resin bound product is stored cold (−20° C.). For analysis, peptide is cleaved from the resin with an acidic cocktail consisting of trifluoroacetic acid (TFA)/H2O/TIPS (triisopropylsilane)/DTT (dithiothreitol) in the following ratio: (0.93v/0.04v/0.03v/0.03w). The resin is swelled with DCM (4-5 mL, 3×30 min) and drained. The cleavage cocktail (4-5 mL) is added to the pre-swelled resin and the suspension is stirred for 2 hr at room temp. The solution is filtered then the resin is washed with a small amount of DCM and combined with the cleavage solution. The resulting solution is poured into 7-10 volumes of cold (0° C.) methyl-tert-butyl ether (MTBE). The suspension is aged for 30 min at 0° C. then the resulting precipitate is centrifuged and the clear solution is decanted. The residue is suspended in the same volume of MTBE, and the resulting suspension is again centrifuged and decanted. After decanting the clear MTBE solution of the precipitated peptide is dried in vacuo at 40° C. overnight.

### Compound 1 Synthesis by Method 1

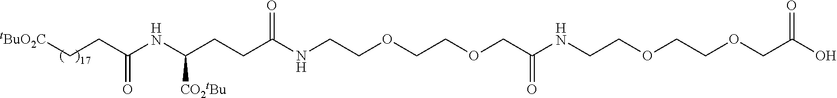

Compound 1 may be prepared substantially by the process described in WO2020/159949.

Dissolve eicosanedioic acid, mono (1,1-dimethylethyl) ester (15.0 kg, limiting reagent) and N-hydroxy-succinimide (1.2 eq) in ethyl acetate at 27° C. Add a solution of

DCC (1.25 eq.) in ethyl acetate and stir the reaction for 24 hr at 22° C. Filter off the resulting DCU by-product and then extract the organic phase three times with 5% NaCl aq. solution. After extraction, concentrate the organic phase, co-evaporate with isopropanol, and then crystallize by addition of heptane. After filtration rinse the filter cake with heptane and dry at 25° C. to afford 17.0 kg of INTI in 87% yield and 99% purity.

Dissolve H-Glu-OtBu (7.7 kg, 1.1 eq) in DCM (54 L) at 20° C., then add a solution of TMSA (11.3 kg) dissolved in DCM (7 L), then stir the reaction mixture for 1 hr at 40° C. Add INT1 (17.0 kg) DCM solution at room temperature and stir 8 hr. After the reaction is complete, DCM is exchanged to ethyl acetate by distillation. Wash the organic phase three times with 2% aq. KHSO4/NaCl aqueous solution then wash 4 times with 2% NaCl aqueous solution. After separation and removal of aqueous phases, concentrate the organic phase with isopropanol, dilute with isopropanol, and then crystallize by addition of water. After filtration, wash the filter cake with a mixture of water/isopropanol, and then dry at 30° C. to produce 17.3 kg of INT 2 in 86% yield and 99% purity.

Dissolve the INT 2 (17.3 kg) and N-hydroxy-succinimide (4.1 kg, 1.2 eq) in ethylacetate (336 kg) at 27° C. Add a solution of DCC (8.33 kg, 1.25 eq) in ethyl acetate and stir the reaction for 24 hr at 22° C. Filter off the resulting DCU by-product. Concentrate the organic phase, co-evaporate with isopropanol, and then crystallize by cooling the isopropanol solution (˜125 L). After, rinse the filter cake with cold isopropanol and dry at 25° C. to afford 16.3 kg INT 3 with 81% yield and 96% purity.

Suspend 17-amino-10-oxo-3,6,12,15 tetraoxa-9-aza heptadecanoic acid (AEEA2) (8.1 kg, 26.3 mol) in DCM (54 L) at 22° C., add a TMSA (7.68 kg, 59.9 mol) solution in DCM (6.2 L), and then stir the reaction mixture for an hour at 40° C. Suspend INT 3 (16 kg) in DCM (31 L) at 35° C. and add to the TMS-protected (AEEA2) mixture at 22° C. Stir the reaction for 12 hr, and after reaction completion the mixture is concentrated, then exchanged to ethyl acetate. Wash the organic phase three times with a 2% aq. KHSO₄/NaCl aqueous solution (˜200 L), and then wash 4 times with a 2% NaCl aqueous solution (˜200 L) to a target pH of 4.5. Concentrate the organic phase and exchange to acetonitrile. Cool the acetonitrile solution to −20° C. and then age the resulting suspension for 15 hr at −20° C. Filter the mixture, rinse the filter cake with cold acetonitrile the dry at <0° C. to afford 18.4 kg of Preparation 6 (88% yield) with 96% purity. Overall yield=53%.

### Compound 1 Synthesis by Method 2

Alternatively, Preparation 6 may be prepared using solid phase peptide synthesis using a peptide synthesizer.

Standard coupling procedures are utilized.

### Standard Coupling Conditions:

0.133 M, 2.0 equiv HATU, 5.0 equiv DIEA, ambient temperature, 3 hours, deprotection for 3×15 min with 20% piperidine/DMF.

### Resin Charging:

FmocNH-AEEA on 2-CTC resin (0.99 mmol/g): 1.01 g in each of the parallel reactions. An automatic program using a DMF swell, followed by Pip/DMF; DMF wash; and amino acid, DIEA, HATU mix; and DMF wash cycles followed by drying.
The resin is cleaved by stirring the combined lots in 30% HFIP/DCM (240 mL) for 1.5 hours. The resin is filtered, washed, and the solvent is removed from the filtrate in vacuo. The resulting oil is dissolved in acetonitrile and solvent is removed again. This operation provides 30.47 g (146% of theoretical yield) of a viscous yellow oil, containing 52.3 area % desired product by UPLC analysis. The crude product is purified by flash chromotagraphy (500 grams of silica gel, eluted with 85% DCM/10% methanol/5% acetic acid, 38×100 mL fractions collected). Previously chromatographed concentrate (17.94 g) is crystallized to yield 13.4 g (74.7% yield), with a UPLC purity of 91.65 area %.

### Example 1

### Example (Linear SPPS)

SEQ ID NO:1 may be prepared substantially by the process described in WO2020/159949 except that Fmoc-L-Lys (mtt)-OH is used instead of Fmoc-L-Lys (IVDde)-OH for the addition of at lysine at position 20 in SEQ ID NO:1:

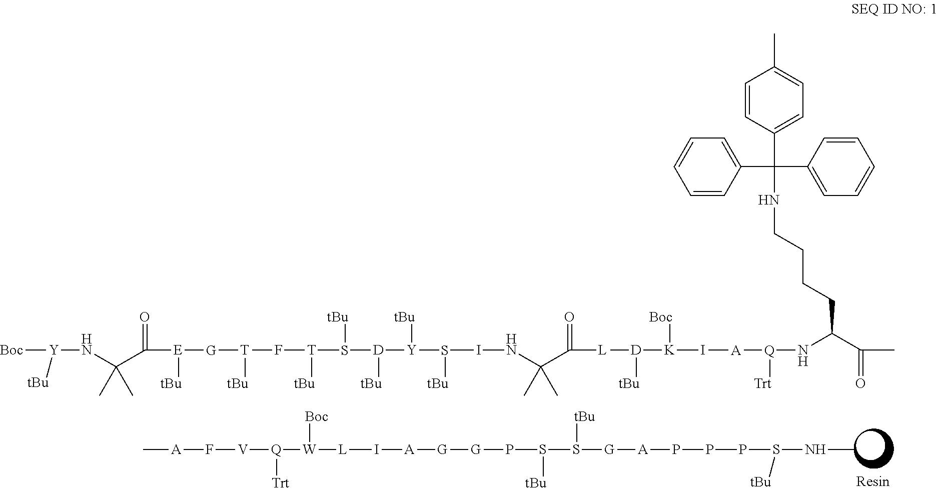

SEQ ID NO:1 (tirzepatide linear 1-39 with Lys-20 mtt protection on resin) (2.0 grams) was suspended in 15 ml of 30% (v/v) HFIP/DCM in a 45 ml Symphony X reactor. The reaction was mixed for 1 hour at ambient temperature and then drained. The resin was again treated with 15 ml of 30% HFIP/DCM, mixed for 1 hour, and drained. The resin was washed five times with 15 ml each of DCM and then dried under vacuum at 35° C. to constant weight to yield SEQ ID NO:2.

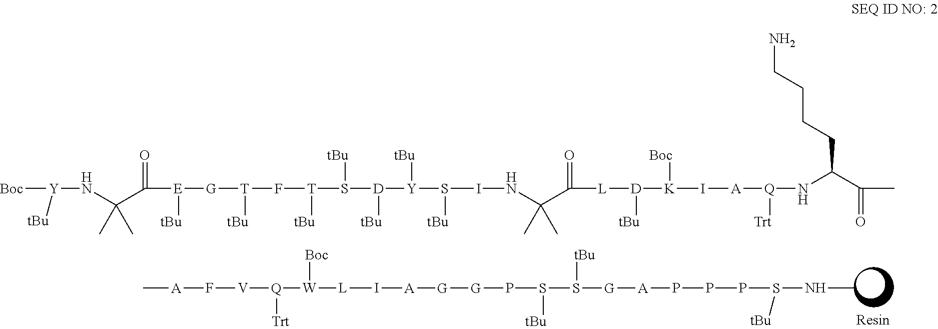

SEQ ID NO:2 (tirzepatide linear 1-39 with Lys-20 NH2 on Sieber resin) (2.0 g) was suspended in 20 ml of 5% (v/v) TFA/DCM and mixed for 30 minutes at ambient temperature. The resin was removed by filtration through a fritted filter and rinsed with 10 ml of DCM. The combined filtrate and wash were treated with 1.05 ml (1 eq based on TFA used) of pyridine to neutralize the TFA. DCM was removed under reduced pressure and the residue was dissolved in 4 ml of DMF. This solution was added in several portions to 20 ml of cold water, which caused the cleaved peptide to precipitate. After cooling at 5° C. for about 30 minutes, the solid was filtered, washed with 20 ml of water, and dried under vacuum at 45° C. to constant weight (1.25 g) to yield SEQ ID NO:3.

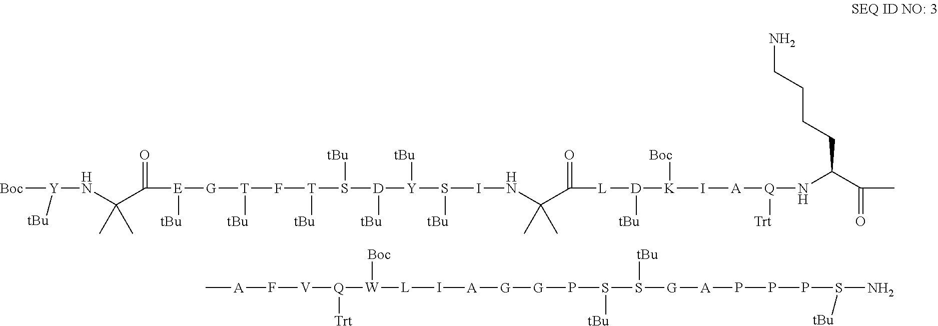

To a solution of SEQ ID NO:3 (tirzepatide 1-39 Lys-20 NH2 linear peptide) (500 mg, 0.091 mmol), Compound 1 (2.0 eq) and 141.3 mg (2.0 eq) of PyBOP in 5 ml of DMF was added DIEA (48 ul, 3.0 eq). The reaction solution was stirred at ambient temperature for 18 hours. Analysis by HPLC showed complete conversion of the linear peptide to desired product. The solution was added in several portions to 40 ml of cold water, which caused a precipitate to form. After cooling at 5° C. for 30 minutes, the solid was collected by filtration on a fritted filter, washed with 20 ml of water, and dried in vacuo to give 535 mg of protected tirzepatide (SEQ ID NO:4.

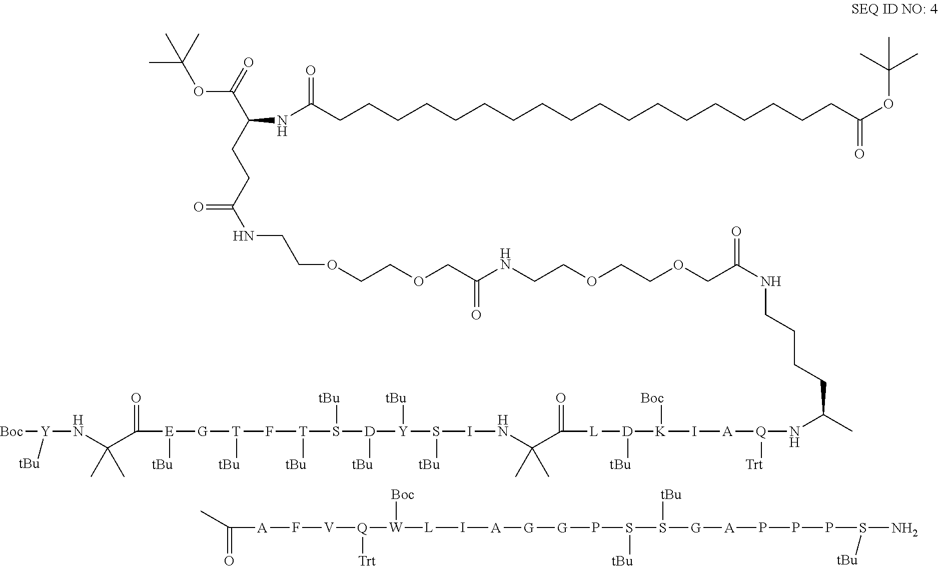

Protected tirzepatide (SEQ ID NO:4) may be deprotected substantially by the process as described in WO2020/159949 to yield tirzepatide (SEQ ID NO:5):

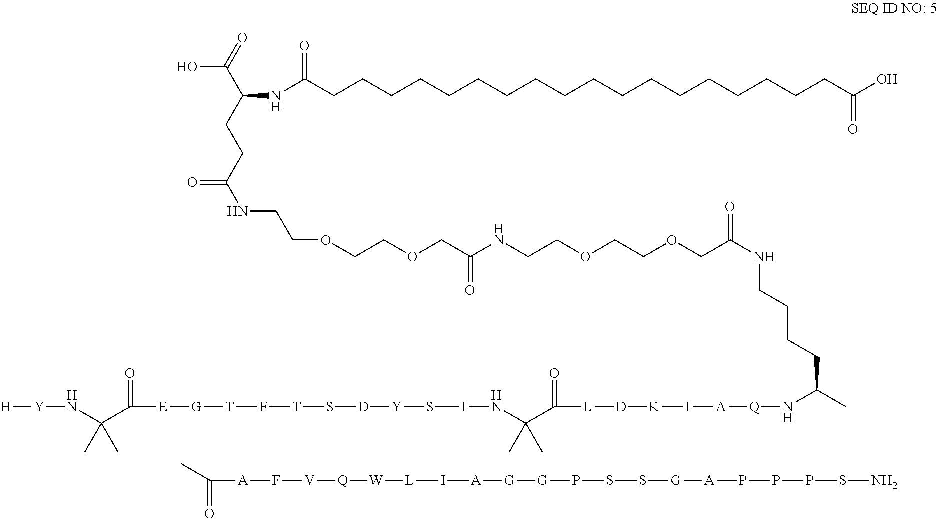

Alternatively, after removal of mtt from SEQ ID NO:1 to form SEQ ID NO:2, compound 1 is coupled to SEQ ID NO:2 to form SEQ ID NO:6.

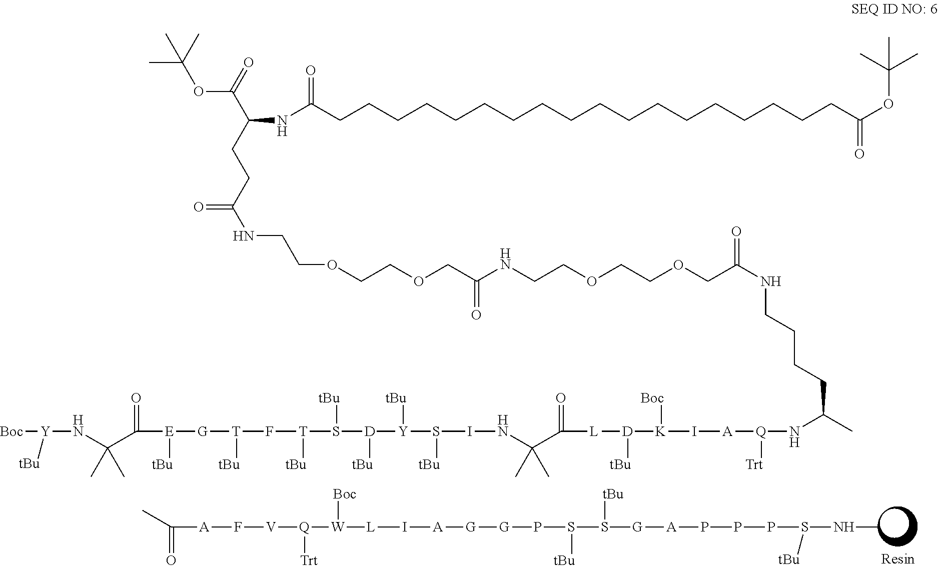

The resin is then removed from SEQ ID NO:6 is to form SEQ ID NO:4, which is globally deprotected to form tirzepatide (SEQ ID NO:5).

### Example 2 MTT Process-2

### Removal of Mtt Group From Lys-20 on TZP 1-39 Linear Peptide:

TZP linear 1-39 with Lys-20 mtt protection on resin (2.0 grams) was suspended in 15 ml of 30% (v/v) HFIP/DCM in a 45 ml Symphony X reactor. The reaction was mixed for 1 hour at ambient temperature and then drained. The resin was again treated with 15 ml of 30% HFIP/DCM, mixed for 1 hour, and drained. The resin was washed five times with 15 ml each of DCM and then dried under vacuum at 35° C. to constant weight.

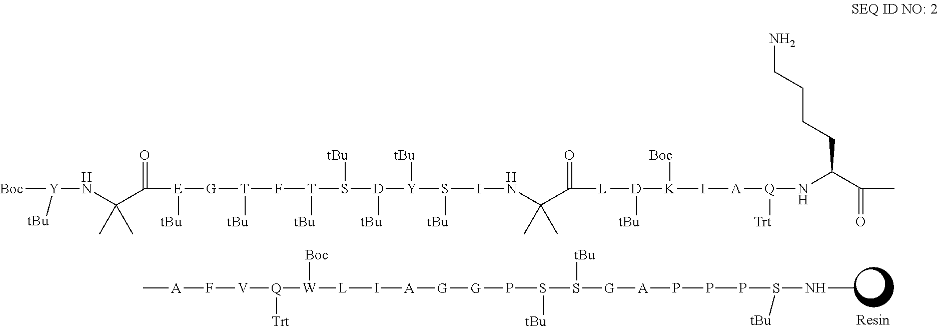

### Soft Cleavage of TZP 1-39 Lys-20 NH₂ Linear Peptide From Sieber Resin:

TZP linear 1-39 with Lys-20 NH2 on Sieber resin (2.0 g) was suspended in 20 ml of 5% (v/v) TFA/DCM and mixed for 30 minutes at ambient temperature. The resin was removed by filtration through a fritted filter and rinsed with 10 ml of DCM. The combined filtrate and wash were treated with 1.05 ml (1 eq based on TFA used) of pyridine to neutralize the TFA. DCM was removed under reduced pressure and the residue was dissolved in 4 ml of DMF. This solution was added in several portions to 20 ml of cold water, which caused the cleaved peptide to precipitate. After cooling at 5° C. for about 30 minutes, the solid was filtered, washed with 20 ml of water, and dried under vacuum at 45° C. to constant weight (1.25 g).

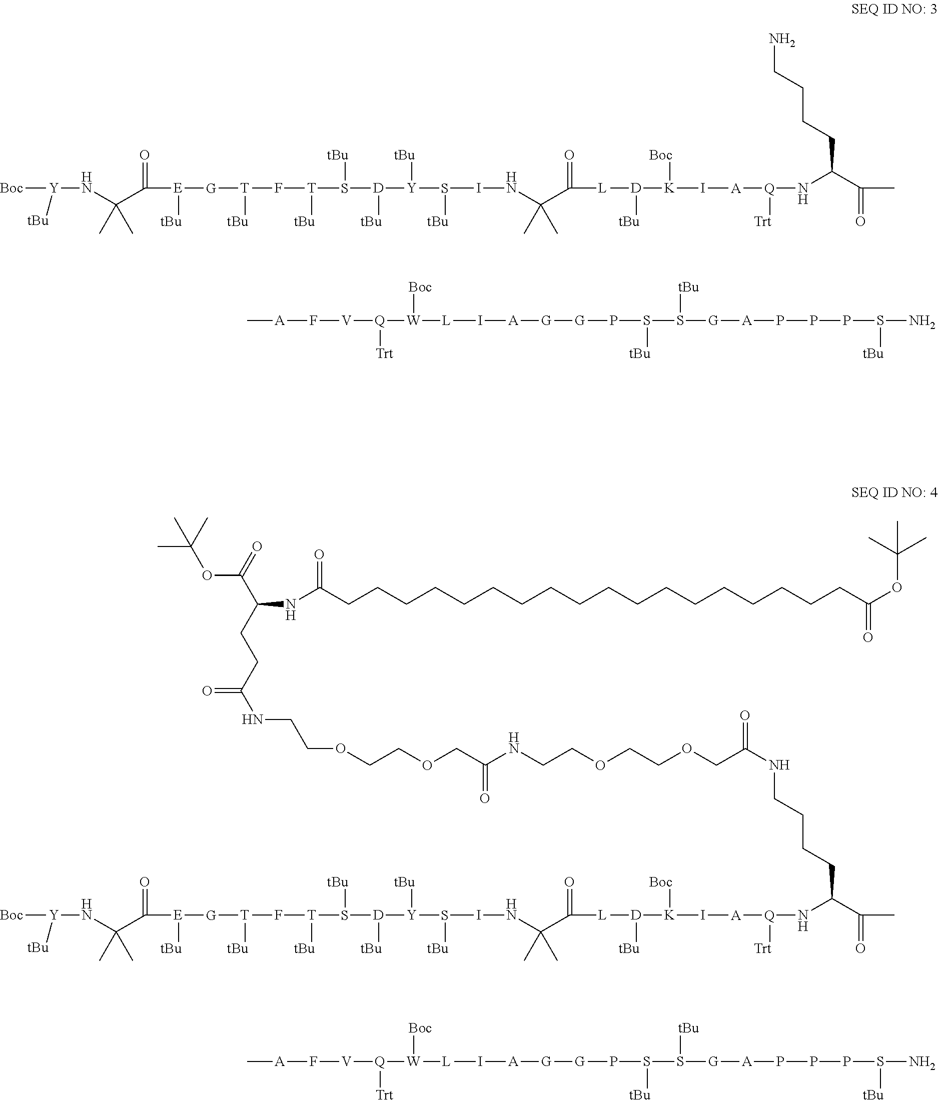

### Coupling of TZP 1-39 Lys-20 NH2 Linear Peptide With (S)-22-(tert-butoxycarbonyl)-45,45-dimethyl-10,19,24,43-tetraoxo-3,6,12,15,44-pentaoxa-9,18,23-triazahexatetracontanoic Acid:

To a solution of TZP 1-39 Lys-20 NH2 linear peptide (500 mg, 0.091 mmol), (S)-22-(tert-butoxycarbonyl)-45,45-dimethyl-10,19,24,43-tetraoxo-3,6,12,15,44-pentaoxa-9,18,23-triazahexatetracontanoic acid (2.0 eq) and 141.3 mg (2.0 eq) of PyBOP in 5 ml of DMF was added DIEA (48 ul, 3.0 eq). The reaction solution was stirred at ambient temperature for 18 hours. Analysis by HPLC showed complete conversion of the linear peptide to desired product. The solution was added in several portions to 40 ml of cold water, which caused a precipitate to form. After cooling at 5° C. for 30 minutes, the solid was collected by filtration on a fritted filter, washed with 20 ml of water, and dried in vacuo to give 535 mg of protected tirzepatide (SEQ ID NO:4).

Protected tirzepatide (SEQ ID NO:4) may be deprotected substantially by the process as described in WO2020/159949 to yield tirzepatide (SEQ ID NO:5)

### Determination of Chiral Purity

The structures of tirzepatide fragments, including amino acid sequences (residue numbers represent position in the fully assembled tirzepatide drug substance), are prepared substantially as described herein. L-and D-isomer fragment materials were synthesized by Eli Lilly and Company (Indianapolis, IN USA) as amorphous solids at high purity. The fragment samples were fully characterized for identity, purity, and content. The tirzepatide fragments are “protected” peptides, where the active side chains of amino acids (such as hydroxyl, carboxyl, amino, etc.) are protected with functional groups such as Fmoc, trityl (Trt), tert-butyloxycarbonyl (Boc), and tertiary butyl (tBu) Chiral purity analyses of the L-isomer fragment materials were also executed using established GCMS and LCMS methodologies (Kalikova et al, Separations 3 pp. 30-48 (2016); Liardon et al, J. Chromatogr. A 203 pp. 385-395 (1981)).

Sequences

SEQ ID NO: 5
Tirzepatide
YX₁EGTFTSDYSIX₂LDKIAQKAFVQWLIAGGPSSGAPPPS

wherein X₁ is Aib; X₂ is Aib; K at position 20 is chemically modified through conjugation to the epsilon-amino group of the K side-chain with (2-[2-(2-Amino-ethoxy)-ethoxy]-acetyl)₂-(γGlu)₁-CO-(CH₂)₁₈-CO₂H; and the C-terminal amino acid is amidated as a C-terminal primary amide

1-8. (canceled)

9. A compound comprising the amino acid sequence of SEQ ID NO: 5, wherein tryptophan at position 25 is oxidized.

10. The compound of claim 9, wherein position 25 is N′-formylkynurenine.

11. A composition comprising: (a) a compound comprising the amino acid sequence of SEQ ID NO: 5; and (b) the compound of claim 9.

12. A method of preventing the formation of the compound of claim 9, the method comprising combining a compound of SEQ ID NO: 8 with phenol.

13. The method of claim 12, wherein the comprises combining a compound of SEQ ID NO: 8 with 1% phenol.
